(19) United States
(12) Patent Application Publication
(10) Pub. No.: US 2025/0170041 A1
(43) Pub. Date: May 29, 2025

(54) USE OF RHAMNOLIPID(S) FOR PREVENTING THE COLOURATION OF CUTANEOUS BLACKHEADS

(71) Applicant: L'OREAL, Paris (FR)

(72) Inventors: ANNE POTTER, Aulnay-Sousbois (FR); NELLY BILLONI, Aulnay-Sousbois (FR); JULIA PUECH, Chevilly-Larue (FR)

(73) Assignee: L'OREAL, Paris (FR)

(21) Appl. No.: 18/838,712

(22) PCT Filed: Feb. 17, 2023
(86) PCT No.: PCT/EP2023/054020

§ 371 Date: Aug. 15, 2024

(30) Foreign Application Priority Data
Feb. 17, 2022 (FR) 2201380

(51) Int. Cl. A61K 8/60; A61Q 19/00

(52) U.S. Cl. CPC A61K 8/602; A61Q 19/00

(57) ABSTRACT

The present invention relates to the cosmetic use of at least one dirhamnolipid of general formula (I): and also the salts thereof, solvates thereof and optical isomers thereof, for reducing and/or inhibiting the development of the colour, for example brown or black colour, of cutaneous comedones and/or preventing the appearance of cutaneous blackheads. It also relates to a non-therapeutic cosmetic method for reducing and/or inhibiting the development of colour, for example brown or black colour, of cutaneous comedones and/or preventing the appearance of cutaneous blackheads, comprising the topical application to the skin of a composition comprising said active agent.

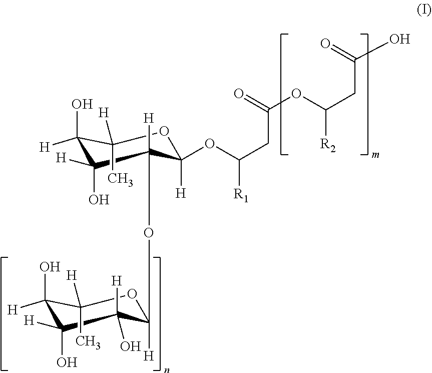

### TECHNICAL FIELD

The present invention relates to the field of cosmetic products, notably intended for preventing cutaneous blackheads and in particular for inhibiting the appearance of the black or brown colour of cutaneous comedones.

More particularly, the present invention is targeted at providing the non-therapeutic cosmetic use of a specific active agent as inhibitor of the black or brown colouration of comedones.

It also relates to a non-therapeutic cosmetic method for reducing and/or inhibiting the development of colour, for example brown or black colour, of cutaneous comedones and/or preventing the appearance of cutaneous blackheads, comprising the topical application to the skin of a composition comprising said active agent.

For the purposes of the present invention, the term “skin” is understood to denote all of the skin of the body, excluding the scalp and the mucous membranes. Preferably, “skin” is targeted at the skin of the face and/or of the hands, in particular the skin of the face, more particularly the skin of the forehead and/or of the alae of the nose and/or of the chin.

### PRIOR ART

There exist cosmetic compositions for combatting the appearance of cutaneous blackheads or for concealing them, in particular on the face. This is because this type of cutaneous imperfection, which relates to all skin typologies, is generally considered to be unattractive and thus undesirable. In view of its black, indeed even brown, colour, it is regarded as excessively visible and all the more so on a lightly pigmented skin.

To date, the source of the colour of the cutaneous blackhead has not been fully established.

It is, on the other hand, known that the cutaneous blackhead results from the process of comedogenesis which affects pilosebaceous units of sebaceous follicle type, which are distributed predominantly on the face.

From a clinical perspective, a cutaneous blackhead is thus an open comedone (non-inflammatory lesion) characterized by distension of the hair canal, sebum retention and excessive cornification of the pilosebaceous duct. Hypercornification would result in sebum retention and in the accumulation of cell debris.

A certain number of solutions for treating cutaneous blackheads have already been provided.

Some of these solutions are based more particularly on the treatment of the blackheads with an active agent capable of preventing, altering or eliminating the blackheads. By way of illustration of these active agents, mention may in particular be made of the active agents capable of dissolving lipids constituting the blackheads, such as the alkyl lactate derivatives described in the patent U.S. Pat. No. 4,540,567, the lipase enzyme, described in the application US 2006/051339, or else neutralized fatty acids as described in the application EP 3 150 187.

However, a number of these active agents have the drawback of detrimentally affecting the organoleptic properties of the composition comprising them, in particular the odour and/or the colour of the composition, over time. These active agents also prove to be effective only with regard to blackheads that have already appeared.

Moreover, other solutions available to date act via mechanical extraction or use laser energy (LED).

As regards the extraction method, this is based on the use, for example, of a comedone suction device, a comedone extractor, or self-adhesive patches to be removed after a few minutes of contact with the skin to be treated. It clearly has the drawbacks of being painful and of generating redness or irritation.

One object of the present invention is very particularly that of providing novel active agents that are very particularly effective with regard to the development of blackheads but are free from the abovementioned drawbacks.

For the purposes of the present invention, the treatment of cutaneous blackheads aims to reduce or even inhibit the development of the black or brown colour of the comedones and therefore to prevent the appearance of the cutaneous blackheads.

### SUMMARY OF THE INVENTION

Thus, according to a first aspect, the present invention relates to the cosmetic use of at least one dirhamnolipid of general formula (I):

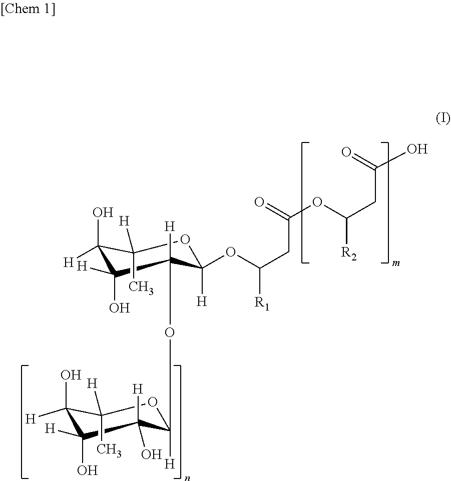

in which:
m=2, 1 or 0,
n=1, and
R₁ and R₂, independently of one another, are identical or different hydrocarbon radicals having from 2 to 24, preferably from 5 to 13 carbon atoms, where appropriate that are branched, optionally substituted, in particular hydroxy-substituted, optionally unsaturated, in particular a mono-, di- or tri-unsaturated alkyl radical,
   and also the salts thereof, solvates thereof and optical isomers thereof,
   for reducing and/or inhibiting the development of the colour, for example brown or black colour, of cutaneous comedones and/or preventing the appearance of cutaneous blackheads.

The invention also relates to a cosmetic method for reducing and/or inhibiting the development of the black or brown colour of comedones, notably for preventing the formation of blackheads, comprising the topical application to the skin of a cosmetic composition comprising at least one dirhamnolipid of general formula (I).

It also relates to a cosmetic method for reducing and/or inhibiting the development of the black or brown colour of cutaneous comedones and/or for preventing the appearance of blackheads, comprising:
a) a step of topical application to the skin of a washing solution;
b) a step of topical application to the skin of a cosmetic composition comprising at least one dirhamnolipid of general formula (I), and
c) preferably, a step of topical application of a composition different from the composition applied in step b) and containing at least one active agent distinct from said dirhamnolipid.

It being possible for steps b) and c) to be carried out simultaneously or successively.

Against all expectations, rhamnolipids have in fact been characterized by the inventors as being capable of inhibiting the black or brown colouration of comedones and hence of preventing the appearance of cutaneous blackheads.

More specifically, and as emerges from the examples below, the inventors have found that dirhamnolipids inhibit the formation of cutaneous blackheads by probably intervening in the process of oxidation of L-DOPA (3,4-dihydroxy-L-phenylalanine). For the record, L-DOPA is involved in the very complex melanogenesis mechanism. More specifically, the oxidation of L-DOPA, promoted by the microbial activity present in the comedone, is expressed by a colouring effect probably of dopachrome type, the latter being a coloured intermediate of the melanin biosynthesis pathway. The dirhamnolipids in accordance with the invention appear to impair the formation of the product of the oxidation reaction in the comedones and thus inhibit the black or brown colouration of the latter. This effectiveness of dirhamnolipids is, to the knowledge of the inventors, characterized for the first time.

Discovered for the first time by Bergström in 1946, rhamnolipids are in fact mainly known as surfactants having a high stability and a high antibacterial and antifungal activity. Thus, they are used for the most part as biosurfactants in cosmetic compositions (US 2014/0296168 A1, EP 2 786 742 A1 and US 2020/069779 A1). More recently, document WO 2020/178048 proposed the use of rhamnolipids as an aid for depositing at least one substance on a surface, such as the hair, and the use of rhamnolipids as active agent in care products for reducing skin reactivity and redness has also been suggested (EP 3 338 763 A1 and EP 3 338 762 A1). However, none of the abovementioned applications targets the use considered by the invention.

It is also important to differentiate the reduction and/or the inhibition of the black or brown colouration of the comedones targeted according to the invention from a skin depigmenting and/or whitening action as described previously. These are clearly two different applications with distinct modes of action. Thus, the depigmentation of the skin is carried out by inhibition of the human melanin biosynthesis pathway, in particular via the inhibition of tyrosinase expression, whereas the inhibition of the brown or black colouration of the comedones is carried out, as mentioned above, by impairing the formation of dopachrome.

More specifically, an “inhibitor of the black or brown colouration of the comedones” is understood to denote, for the purposes of the present invention, a compound capable of slowing down, indeed even inhibiting, the appearance of the black or brown colour of the comedones. Notably, a compound is considered an inhibitor of the black or brown colouration of the comedones if the percentage of inhibition with regard to the development of the colour is at least 15%, preferably at least 20%, more particularly at least 25%, in particular as evaluated according to the protocol detailed in the examples below.

The cosmetic uses and methods considered according to the invention are nontherapeutic.

Preferably, the skin is the skin of the face and/or of the body, in particular of the face and/or of the hands, preferably of the face, and more particularly of the forehead and/or of the alae of the nose and/or of the chin.

According to a first variant, the skin is oily and/or shiny skin, in particular skin exhibiting excessive secretion and excretion of sebum. According to this variant, the skin is in particular oily and/or shiny skin, notably of phototype 1, 2, 3 or 4, in particular oily and/or shiny skin of the face and/or of the hands, more particularly oily and/or shiny skin of the forehead and/or of the alae of the nose and/or of the chin.

According to a second variant, the skin is healthy skin devoid of the clinical manifestations of acne, namely acne lesions, such as the presence of numerous acne pimples. This is because the blackheads constitute cutaneous imperfections of the skin, distinct from ailments of the skin, such as acne. In particular, the blackhead is not an inflammatory lesion of the skin, in contrast to the lesions characteristic of acne. Furthermore, the regions of the face which are conducive to the development of blackheads, namely more particularly the alae of the nose, are precisely not regions that favour the development of acne.

### DETAILED DESCRIPTION

### Rhamnolipids

Rhamnolipids are glycolipids produced by various bacterial species. They consist of one rhamnose fragment (monorhamnolipid) or of two rhamnose fragments (dirhamnolipid) linked by a glycosidic bond to one, two or three chains of β-hydroxylated fatty acids linked to one another by an ester bond.

More specifically, these monorhamnolipids and dirhamnolipids correspond to the following general formula:

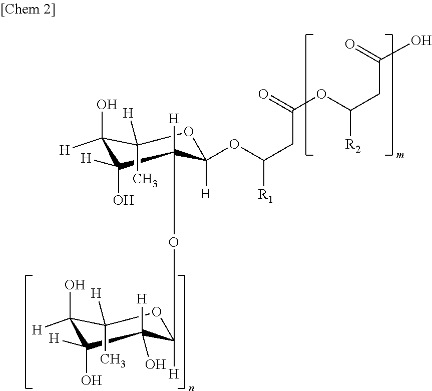

in which:
m=2, 1 or 0,
n=1 or 0, and
R₁ and R₂, independently of one another, are identical or different hydrocarbon radicals having from 2 to 24, preferably from 5 to 13 carbon atoms, where appropriate that are branched, optionally substituted, in particular hydroxy-substituted, optionally unsaturated, in particular a mono-, di- or tri-unsaturated alkyl radical.

Thus, when n is equal to 0, the general formula (I) protects monorhamnolipids and, when n is equal to 1, it protects dirhamnolipids.

As regards the present invention, it very particularly targets the use of at least one dirhamnolipid of general formula (I):

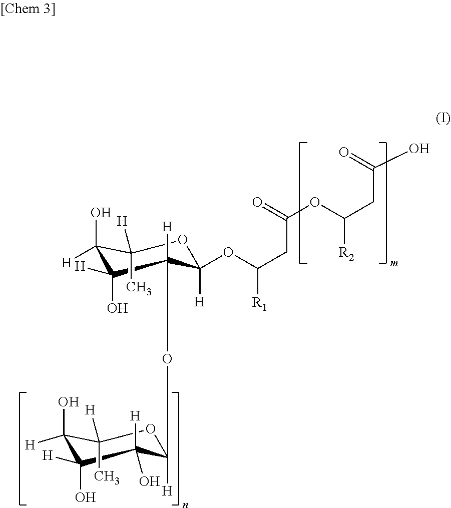

in which:
m=2, 1 or 0;
n=1;
R₁ and R₂, independently of one another, are identical or different hydrocarbon radicals having from 2 to 24, preferably from 5 to 13 carbon atoms, where appropriate that are branched, optionally substituted, in particular hydroxy-substituted, optionally unsaturated, in particular a mono-, di- or tri-unsaturated alkyl radical;
   and also the salts thereof, solvates thereof and optical isomers thereof,
   for reducing and/or inhibiting the development of the colour, for example brown or black colour, of cutaneous comedones and/or preventing the appearance of cutaneous blackheads.

In so far as n is equal to 1 in the general formula (I), the corresponding compounds that are suitable for the invention are also referred to as dirhamnolipids in the present description.

The glycosidic bond between the two rhamnose fragments may be in the alpha or beta configuration and is preferably in the alpha configuration.

In the context of the invention,
the salts of the dirhamnolipids of formula (I) are more particularly the carboxylate salts thereof with an organic or inorganic cation and notably with a cation chosen from sodium, potassium, calcium and ammonium,
the solvated forms of the dirhamnolipids of formula (I) are more particularly those solvated with one or more molecules of water or of organic solvents, for example a hydrate or a solvate of a linear or branched alcohol, such as ethanol or isopropanol,
   the optically active carbon atoms of the fatty acids are preferably in the form of R enantiomers, and
an “alkyl” radical, a saturated, linear or branched aliphatic group; for example, a C₁-C₂₀ alkyl group represents a linear or branched hydrocarbon chain of 1 to 20 carbon atoms, more particularly a methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl or eicosyl.

In particular, the use according to the invention uses at least one dirhamnolipid of general formula (I), in which R₁ and R₂, which may be identical or different, are chosen from pentenyl, hexenyl, heptenyl, octenyl, nonenyl, decenyl, undecenyl, dodecenyl and tridecenyl radicals and radicals of formula —(CH₂)ₒCH₃ with o being an integer varying from 1 to 23, in particular from 3 to 15 and more particularly from 4 to 12.

According to one variant of the invention, the present invention targets the use of at least one dirhamnolipid of general formula (I) in which m is equal to 1.

According to one variant of the invention, the claimed use uses said dirhamnolipid of general formula (I) in the form of a mixture of at least two and preferably of at least three dirhamnolipids of general formula (I) in which m is preferably equal to 1.

According to another variant of the invention, said dirhamnolipid of formula (I) is used in the form of a mixture comprising at least one monorhamnolipid.

More particularly, the claimed use uses at least one dirhamnolipid compound, of formula (II):

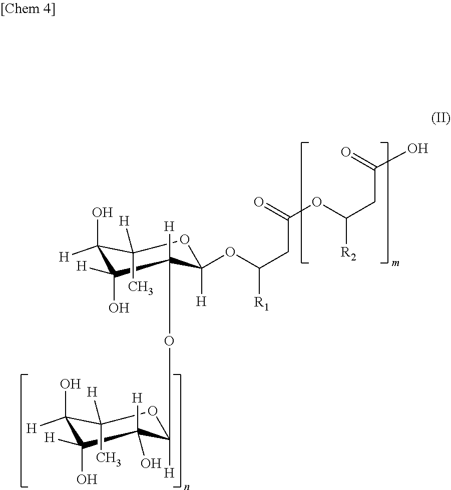

in which:
m=2, 1 or 0, and preferably m=1,
n=1,
R₁ is a —(CH₂)ₚ—CH₃ radical, with p being an integer varying from 1 to 23, preferably from 4 to 12,
R₂ is a —(CH₂)_{q}—CH₃ radical, with q being an integer varying from 1 to 23, preferably from 4 to 12,
   and also the salts thereof, solvates thereof and optical isomers thereof.

As non-limiting illustrations of the dirhamnolipids of formula (II) that may be suitable for the invention, mention may notably be made of the compounds of formula Di-RL-CXCY, as defined in Table 1 below.

The formula diRL-CXCY is an alternative way of writing in order to represent a dirhamnolipid (diRL) functionalized by two radicals R₁ and R₂ respectively represented by the symbols CX and CY, the integers X and Y being respectively equal to p+4 and q+4.

TABLE 1

| Compounds | Di-RL-CXCY | p | q |
|---|---|---|---|
| 1 | diRL-C8C8 | 4 | 4 |
| 2 | diRL-C8C10 | 4 | 6 |
| 3 | diRL-C10C8 | 6 | 4 |
| 4 | diRL-C10C10 | 6 | 6 |
| 5 | diRL-C10C12 | 6 | 8 |
| 6 | diRL-C12C10 | 8 | 6 |
| 7 | diRL-C12C12 | 8 | 8 |
| 8 | diRL-C12C14 | 8 | 10 |
| 9 | diRL-C14C12 | 10 | 8 |
| 10 | diRL-C14C14 | 10 | 10 |
| 11 | diRL-C14C16 | 10 | 12 |
| 12 | diRL-C16C14 | 12 | 10 |
| 13 | diRL-C16C16 | 12 | 12 |

Advantageously, the dirhamnolipid used according to the invention is at least one dirhamnolipid of formula (II), in which p and q are identical and equal to 6 and m is equal to 1, also referred to as di-RL-C10C10, or one of the salts, solvates and optical isomers thereof.

According to one advantageous embodiment, this dirhamnolipid of formula (II) in which p and q are identical and equal to 6 and m is equal to 1 is used in the form of a mixture of rhamnolipids, comprising it in a proportion of at least 50% by weight, and preferably from 51% to 85% by weight, relative to the total weight of rhamnolipids.

According to another preferred embodiment, the dirhamnolipid used according to the invention is at least one dirhamnolipid of formula (II), in which m is equal to 1, p is equal to 6 and q is equal to 8, or one of the salts, solvates and optical isomers thereof.

According to another advantageous embodiment, the dirhamnolipid used according to the invention is a dirhamnolipid of general formula (I) in which m is equal to 1, R₁ is a —(CH₂)ₒCH₃ radical, with o being an integer varying from 4 to 12, and R₂ is chosen from pentenyl, hexenyl, heptenyl, octenyl, nonenyl, decenyl, undecenyl, dodecenyl and tridecenyl radicals, and preferably R₁ is a —(CH₂)₆CH₃ radical and R₂ is a nonenyl radical, or one of the salts, solvates and optical isomers thereof.

According to another preferred embodiment, the dirhamnolipid considered according to the invention is used in the form of a mixture of rhamnolipids comprising at least two, in particular at least three dirhamnolipids of formula (II) or of formula (I) chosen from:
a dirhamnolipid of formula (II), in which p and q are identical and equal to 6 and m is equal to 1,
a dirhamnolipid of formula (II) in which m is equal to 1, p is equal to 6, q is equal to 8, and
a dirhamnolipid of general formula (I) in which m and n are equal to 1, R₁ is a —(CH₂)ₒCH₃ radical with o being an integer varying from 4 to 12, and R₂ is chosen from pentenyl, hexenyl, heptenyl, octenyl, nonenyl, decenyl, undecenyl, dodecenyl and tridecenyl radicals, and preferably, R₁ is a —(CH₂)₆CH₃ radical and R₂ is a nonenyl radical.

Very particularly advantageously, the dirhamnolipid considered according to the invention is used in the form of a mixture of rhamnolipids comprising at least two, in particular at least three dirhamnolipids of formula (II) or of formula (I) chosen from:
at least 50% by weight, preferably from 51% to 85% by weight, of a dirhamnolipid of formula (II) in which p and q are identical and equal to 6 and m is equal to 1, relative to the total weight of rhamnolipids,
from 0.5% to 25% by weight, preferably from 5% to 15% by weight, of a dirhamnolipid of formula (II) in which p is equal to 6, q is equal to 8 and m is equal to 1, relative to the total weight of rhamnolipids, and
from 0.5% to 15% by weight, preferably from 3% to 12% by weight, preferably from 5% to 10% by weight, of a dirhamnolipid of general formula (I) in which n and m are equal to 1, R₁ is a —(CH₂)₆CH₃ radical and R₂ is a nonenyl radical, relative to the total weight of rhamnolipids.

As specified above, rhamnolipids are customarily prepared by processes known to those skilled in the art starting from producing bacteria, such as Pseudomonas.

Appropriate fermentation methods are reviewed in D. Haferburg, R. Hommel, R. Claus and H. P. Kleber in Adv. Biochem. Ing./Biotechnol. (1986) 33, 53-90 and by F. Wagner, H. Bock and A. Kretschmar in Fermentation (ed. R. M. Lafferty) (1981), 181-192, Springer Verlag, Vienna.

Use may be made, as dirhamnolipids, of the one sold under the name Rheance One by Evonik (INCI name: glycolipids).

Advantageously, said dirhamnolipid of general formula (I) or one of the mixtures thereof notably as detailed above is used in the form of a topical cosmetic composition containing it. Such a composition preferably contains from 0.01% to 10% by weight, preferably from 0.1% to 5% by weight, even better still from 0.3% to 3% by weight of dirhamnolipid(s) of general formula (I) relative to the total weight of the cosmetic composition.

### Composition

In addition to said dirhamnolipid, a composition according to the invention further comprises a physiologically acceptable medium.

Such a medium is considered to be physiologically acceptable when it does not cause any tingling, tautness or discomfort unacceptable for the user.

A composition used according to the invention can be provided in any formulation form normally used in the cosmetics field.

It can in particular be in the form of an aqueous or aqueous/alcoholic solution, which is optionally gelled, of a dispersion of the lotion type, which is optionally a two-phase lotion, of an oil-in-water or water-in-oil or multiple emulsion, of an aqueous gel, of a dispersion of oils in an aqueous phase, in particular by means of spherules, it being possible for these spherules to be polymer particles or, better still, lipid vesicles of ionic and/or non-ionic type, or else in the form of a powder, of a serum, of a paste or of a flexible rod or also of a stick.

It can be of solid, pasty or more or less fluid liquid consistency.

According to a preferred variant, the composition considered according to the invention differs from compositions having an essentially detergent purpose with regard to the skin, hair and/or mucous membranes, such as soaps, shampoos and shower gels for washing and/or cleansing.

A composition according to the invention can comprise any constituent normally employed in the topical application and administration envisaged.

A composition according to the invention can advantageously be provided in the form of an emulsion, in particular obtained by dispersion of an aqueous phase in a fatty phase (W/O) or of a fatty phase in an aqueous phase (O/W), of liquid or semi-liquid consistency of the milk type, or of soft, semi-solid or solid consistency of the cream or gel type, or also of multiple emulsion (W/O/W or O/W/O). These compositions are prepared according to the usual methods.

More particularly, this composition is intended for topical application and preferably is in the form of an oil-in-water emulsion. Preferably, such an emulsion is not intended to be rinsed off after application.

A composition according to the invention is intended to be applied on skin.

Preferably, the skin is the skin of the face and/or of the body, in particular of the face and/or of the hands, preferably of the face, and more particularly of the forehead and/or of the alae of the nose and/or of the chin.

According to a first variant, the skin is oily and/or shiny skin, in particular skin exhibiting excessive secretion and excretion of sebum. According to this variant, the skin is in particular oily and/or shiny skin, notably of phototype 1, 2, 3 or 4, in particular oily and/or shiny skin of the face and/or of the hands, more particularly oily and/or shiny skin of the forehead and/or of the alae of the nose and/or of the chin.

According to a second variant, the skin is healthy skin devoid of the clinical manifestations of acne, namely acne lesions, such as the presence of numerous acne pimples. This is because the blackheads constitute cutaneous imperfections of the skin, distinct from ailments of the skin, such as acne. In particular, the blackhead is not an inflammatory lesion of the skin, in contrast to the lesions characteristic of acne. Furthermore, the regions of the face which are conducive to the development of blackheads, namely more particularly the alae of the nose, are precisely not regions that favour the development of acne.

A composition used according to the invention can advantageously comprise at least one additional active agent.

It can in particular be at least one active agent for caring for oily skin.

It can also advantageously comprise at least one additional active agent for caring for aged skin.

The expression “additional active agent” is understood to mean, in the context of the present invention, a compound which has, by itself, that is to say not requiring the intervention of an external agent in order to activate it, a biological activity which can in particular be:
a desquamating activity (which makes possible the opening of the comedones), and/or
an antimicrobial activity (in particular with regard to C. acnes), and/or
a soothing or anti-irritant activity, and/or
a sebum-regulating or anti-seborrheic activity, and/or
an astringent activity, and/or
an activity intended to combat and/or prevent the signs of ageing, in particular chronobiological ageing, for example wrinkles and fine lines.

The additional active agent which can be used in the compositions of the invention is preferentially chosen from desquamating agents, soothing agents, anti-irritant agents, sebum-regulating or anti-seborrhoeic agents, astringent agents and mixtures thereof in any proportions.

The additional active agent for caring for oily skin used in the composition according to the invention can represent from 0.0001% to 20%, preferably from 0.01% to 10% and better still from 0.01% to 5% by weight, relative to the total weight of the composition.

Moreover, a composition used according to the invention may advantageously comprise from 5% to 80% by weight and preferably from 35% to 75% by weight of water, relative to the total weight of said composition.

The pH of said composition is advantageously less than or equal to 8, preferably ranging from 4 to 7, even better still ranging from 4.5 to 6.5.

Of course, a person skilled in the art will take care to choose the optional additional compound(s), and/or the amount thereof, in such a way that the advantageous properties of the compound(s) of general formula (I) according to the invention are not, or not substantially, adversely affected by the envisaged addition and that the properties of the compositions resulting therefrom are compatible with the topical administration route.

A composition of the invention can advantageously exhibit a firm and compact feel when taken up. It can be thick on application and subsequently be transformed, melt and release freshness.

As specified, it is advantageously in the form of an oil-in-water emulsion, advantageously which is very fluid.

A composition can alternatively have the form of a product for caring for or making up the face and/or the body, and be packaged, for example, in the form of cream in a jar or of fluid in a tube or in a pump-action bottle or in a dropper bottle.

A composition according to the invention can be manufactured by any known process generally used in the cosmetics field.

The ingredients are mixed before they are shaped, in the order and under conditions which are easily determined by a person skilled in the art.

According to a specific form of the invention, other agents intended to make the appearance and/or the texture of the skin more attractive can also be added to the composition according to the invention.

### Cosmetic Methods

As mentioned above, the present invention relates to a cosmetic method for reducing and/or inhibiting the development of the black or brown colouration of comedones, notably for preventing the formation of blackheads, comprising the topical application to the skin of a cosmetic composition comprising at least one dirhamnolipid of general formula (I).

The cosmetic method of the invention is carried out by administering, by the topical route, a composition comprising at least one dirhamnolipid of general formula (I), preferably in a physiologically acceptable medium.

The topical administration consists of the external application to the skin of cosmetic compositions according to the usual techniques for use of these compositions.

By way of illustration, the cosmetic method according to the invention can be carried out by topical application, for example daily, of at least one dirhamnolipid of general formula (I) and notably one of the mixtures thereof in particular as detailed above, which can, for example, be formulated in the form of a cream, gel, serum, lotion, emulsion, makeup-removing milk, stick or aftersun composition, preferably in the form of an emulsion.

According to one embodiment, the application is repeated, for example, 1 to 2 times daily for one day or more and generally over an extended period of at least 4 weeks, indeed even 4 to 15 weeks, with, if appropriate, one or more periods of interruption.

According to another embodiment, the application is daily (once per day) and generally over an extended period of at least 4 weeks, indeed even 4 to 15 weeks, with, if appropriate, one or more periods of interruption.

According to another embodiment, the composition including at least one dirhamnolipid of general formula (I) or one of the mixtures thereof as defined above will be applied to skin regions which have been cleansed beforehand, for example using an appropriate soap, and/or cleared beforehand of blackheads, for example using an adhesive patch or a mechanical action.

According to one embodiment variant, the composition containing at least one dirhamnolipid as defined previously, will be applied to skin regions which have been cleansed beforehand using a washing solution, where appropriate itself comprising at least one rhamnolipid, and notably a dirhamnolipid.

Furthermore, treatment combinations with optionally topical forms, in order to complement or to reinforce the activity of the dirhamnolipid of general formula (I), can be envisaged.

It might be possible to imagine a topical treatment with a composition containing at least one dirhamnolipid of general formula (I), combined with a supplementary composition dedicated to a topical application and containing at least one active agent distinct from the dirhamnolipid of general formula (I).

Thus, the present invention relates in particular to a cosmetic method for reducing and/or inhibiting the development of the black or brown colour of cutaneous comedones and/or for preventing the appearance of blackheads, comprising:
a) a step of topical application to the skin of a washing solution;
b) a step of topical application to the skin of a cosmetic composition comprising at least one dirhamnolipid of general formula (I) or (II) as defined above, notably in the form of an oil-in-water emulsion; and
c) preferably, a step of topical application of a composition different from the composition applied in step b) and containing at least one active agent distinct from the dirhamnolipid of general formula (I) or (II),
   it being possible for steps b) and c) to be carried out simultaneously or successively.

Such a composition applied in step c) can be described as “supplementary composition”.

The skin considered in the method is the skin of the face and/or of the body, in particular of the face and/or of the hands, preferably of the face, and more particularly of the forehead and/or of the alae of the nose and/or of the chin.

The washing solution may notably be in various forms, such as a solution, an aqueous solution, a lotion, a milky lotion, a cream, gel, a liquid gel, a paste, a serum, a suspension, a dispersion, a fluid, a milk, an emulsion (O/W or W/O form), or others, preferably in the form of a solution or an aqueous gel.

In one embodiment variant, the washing composition of step a) may contain a dirhamnolipid of general formula (I), and notably at least one dirhamnolipid of general formula (I), or even of formula (II), or better still of formula (II) in which p and q are identical and equal to 6.

As regards the supplementary composition, which is different from the composition used according to the invention as it is devoid of dirhamnolipid of formula (I), it may be in the form of an emulsified oil-in-water gel, comprising at least one polyol; at least one hydrophilic gelling agent; at least one derivative of polyethylene glycol and of mono-, di- and triglycerides of an acid comprising at least one alkyl chain ranging from C₆ to C₁₆, and having at least two ethylene oxide groups; at least one non-volatile ether oil having from 10 to 40 carbon atoms; and at least one non-volatile monoester oil.

Throughout the description, including the claims, the expressions “between . . . and . . . ”, “of between . . . and . . . ” and “ranging from . . . to . . . ” should be understood as meaning limits included, unless otherwise specified.

The examples which follow illustrate the present invention without limiting the scope thereof.

### EXAMPLE

### Methodology on a Reconstructed Epidermis for Characterization of the Appearance of Dark-Coloured Structures and its Development Over Time

For the purposes of the present invention, the term “dark-coloured structures” is understood to denote the structures responsible for the colouration of blackheads.

This method is targeted at characterizing the change over time, in the expression of colour, brought about by an intracellular extract of bacteria of Cutibacterium acnes type (formerly listed under the name Propionibacterium acnes) in the presence of 3,4-dihydroxy-L-phenylalanine (L-DOPA), which extract is deposited at the surface of an EpiSkin™ reconstructed epidermis.

To do this, an intracellular extract of bacteria of Cutibacterium acnes CIP 53.117 type, stored at 4° C. in lyophilized form, is suspended in 1×PBS (Dulbecco's Phosphate Buffered Saline, supplied by Gibco BRL, Life Technologies) at a concentration of 6 mg/ml. This extract is subsequently deposited at the surface of an EpiSkin epidermis in the presence of 3,4-dihydroxy-L-phenylalanine (L-DOPA—supplied by Sigma-Aldrich) at 1 mg/ml.

The expression of a colouring effect and its development towards a dark colour are observed visually, starting from 48 hours of incubation.

### In Vitro Methodology Used for the Evaluation of the Ability to Inhibit the Development of the Colour

This method is based on the measurement of absorbance, over time, carried out directly on a composition formed of a mixture of intracellular extract of bacteria of Cutibacterium acnes type, of 3,4-dihydroxy-L-phenylalanine (L-DOPA) and of the compound to be tested.

The measurements are compared with those carried out for a mixture of an intracellular extract of bacteria of Cutibacterium acnes type and of 3,4-dihydroxy-L-phenylalanine (L-DOPA), in the absence of compound to be tested (reference).

The method is adapted from a method for the enzymatic assay of the tyrosinase activity (Behbahani et al., Microchemical Journal (1993), 47, 251), which is based on the absorbance properties at 475 nm of a coloured intermediate of the pathway for the biosynthesis of melanin starting from L-DOPA, namely dopachrome, a product of the oxidation of L-DOPA.

### Preparation of the Reference Composition

An intracellular extract of bacteria of Cutibacterium acnes CIP 53.117 type, stored at 4° C. in lyophilized form, is suspended in 1×PBS (supplied by Gibco BRL, Life Technologies) at a concentration of 6 mg/ml.

A solution of L-DOPA at a concentration of 0.5 mg/ml is prepared by bringing together L-DOPA (supplied by Sigma-Aldrich) and milliQ water, so as to have an L-DOPA concentration of 1 mg/ml, and then diluting with 1×PBS (Dulbecco's Phosphate Buffered Saline, supplied by Gibco BRL, Life Technologies).

The mixture of 50 μl of these two solutions and of 100 μl of 1×PBS (Dulbecco's Phosphate Buffered Saline supplied by Gibco BRL, Life Technologies) results in a composition with a total volume of 200 μl containing L-DOPA (at a concentration of 125 μg/ml) and the Cutibacterium acnes extract (at a concentration of 1.5 mg/ml).

### Preparation of a Composition 1 Comprising Only a Cutibacterium acnes Extract

The 6 mg/ml Cutibacterium acnes extract was diluted with 1×PBS (Dulbecco's Phosphate Buffered Saline supplied by Gibco BRL, Life Technologies) so as to form a 200 μl composition with a concentration of 1.5 mg/ml of Cutibacterium acnes.

### Preparation of a Composition 2 Comprising Only L-DOPA

The 0.5 mg/ml L-DOPA solution was also diluted with 1×PBS (Dulbecco's Phosphate Buffered Saline supplied by Gibco BRL, Life Technologies) so as to form a 200 μl composition with a concentration of 125 μg/ml of L-DOPA.

### Absorbance Measurement

The compositions to be tested and the reference composition are introduced separately into wells of a 96-well microplate suitable for reading in visible spectrophotometry, and placed at a temperature of 32° C.

The formation of a coloured product is then monitored over time by spectrophotometry. To do this, the absorbance, or optical density (O.D.), is measured at 475 nm from the time 0 to the time 2 hours by means of a Spectra Max M5^{e} reader and the Spectra Max software (the time 0 corresponds to the addition of composition 1 or the introduction of the composition into the wells).

The formation of a coloured product for the reference composition, oxidation product of L-DOPA, is visually observed.

However, no appearance of a significant colouring effect over time is noted for compositions 1 and 2 comprising solely a Cutibacterium acnes extract and solely L-DOPA, respectively.

This confirms the catalytic activity of the bacterial extract of Cutibacterium acnes on the development of the dark colour by oxidation of L-DOPA.

It is thus possible to monitor the formation of the coloured intermediate of the melanin biosynthesis pathway by virtue of its properties of absorption at 475 nm. The effectiveness of a compound in preventing the development of colour of cutaneous blackheads can be evaluated by this means.

### Parameters Studied for the Evaluation of the Effectiveness of the Various Compounds to be Tested

The ability to inhibit the development of colour of a given compound was assessed by comparing the absorbance measurements obtained for the test compositions with those obtained for the reference composition.

More specifically, the inhibitory effect of the compound to be tested was evaluated by comparing the initial rate of oxidation of the L-DOPA under the action of the bacterial extract at 475 nm for the test composition with that of the reference composition in order to obtain a corresponding percentage of inhibition.

In practice, the initial rate (Vi) of the oxidation reaction is calculated from the linear part of the curve of the graph of the absorbance values. As this part lies between 10 and 40 minutes, the initial rate of the oxidation reaction is determined using the following relationship:

$$Vi = \frac{\begin{matrix}O . D . measured at 40 minutes - \\ O . D . measured at 10 minutes\end{matrix}}{30 minutes} \qquad [ Math 1 ]$$

The percentage of inhibition (% inhib) of a given compound with regard to the formation of the coloured product is subsequently obtained using the values of the initial rate of the oxidation reaction for the reference composition (Vi_{ref}) and for the test composition (Viₜₑₛₜ) according to the following relationship:

$$\% inhib = \frac{{Vi}_{ref} - {Vi}_{test}}{{Vi}_{ref}} \times 100 \qquad [ Math 2 ]$$

For the purposes of the invention, a compound is considered to have an inhibitory effect if the % inhib value is at least 15%.

### Example 1—In Vitro Evaluation of the Ability of a Dirhamnolipid in Accordance With the Invention to Inhibit the Development of the Colour

Three test compositions, 1, 2, and 3, comprising at least one dirhamnolipid of general formula (I), RL, (reference RheanceOne® from Evonik) at respective concentrations of 1.25, 2 and 2.5 mg/ml were prepared. These compositions, with a total volume of 200 μl, also contain L-DOPA (25 μg), the Cutibacterium acnes extract (0.3 mg) and the PBS buffer which are mentioned in the methodology part above. The change in the optical density at 475 nm of these three compositions, and also the reference composition, is then measured according to the protocol indicated in the methodology part presented above.

The measured optical density values are collated in the following table.

TABLE 2

| | Time (in minutes) | | | | | | | | |
|---|---|---|---|---|---|---|---|---|---|
| | 0 | 10 | 20 | 30 | 40 | 60 | 80 | 100 | 120 |
| Reference | 0.0440 | 0.0450 | 0.0470 | 0.0485 | 0.0505 | 0.0536 | 0.0556 | 0.0567 | 0.0567 |
| composition | | | | | | | | | |
| OD value | | | | | | | | | |
| Test 1 composition | 0.0441 | 0.0451 | 0.0465 | 0.0478 | 0.0504 | 0.0532 | 0.0563 | 0.0579 | 0.0592 |
| (1.25 mg/ml RL) | | | | | | | | | |
| OD value | | | | | | | | | |
| Test 2 composition | 0.0434 | 0.0440 | 0.0451 | 0.0460 | 0.0483 | 0.0504 | 0.0532 | 0.0546 | 0.0570 |
| (2 mg/ml RL) | | | | | | | | | |
| OD value | | | | | | | | | |
| Test 3 composition | 0.0433 | 0.0441 | 0.0453 | 0.0462 | 0.0475 | 0.0500 | 0.0519 | 0.0534 | 0.0548 |
| (2.5 mg/ml RL) | | | | | | | | | |
| OD value | | | | | | | | | |

The percentage of inhibition values, calculated from the mathematical formulae detailed in the methodology part above, are presented in Table 3 below.

TABLE 3

| | Test 1 | Test 2 | Test 3 |
|---|---|---|---|
| | composition | composition | composition |
| Composition | (1.25 mg/ml RL) | (2 mg/ml RL) | (2.5 mg/ml RL) |
| % inhibition | 3.6% | 21.8% | 38.18% |

An effectiveness of the dirhamnolipid of general formula (I) which increases with the concentration of RL, up to more than 38% with an RL concentration of 2.5 mg/ml, is effectively observed.

### Example 2—Cosmetic Compositions Comprising at Least One Dirhamnolipid in Accordance With the Invention

A composition for topical application is prepared which comprises:

TABLE 4

| Compound (INCI name) | | |
|---|---|---|
| Xanthan gum | 0.5% | by weight |
| Glycerol | 2% | by weight |
| Phenoxyethanol | 0.5% | by weight |
| Dirhamnolipid of general formula (I) | 1% | by weight |

| | |
|---|---|
| (RheanceOne ® from Evonik) | |
| Water | qs 100% |

The composition applied to the skin makes it possible to inhibit the appearance of the black or brown colouration of the comedones.

1. Cosmetic use of at least one dirhamnolipid of general formula (I): 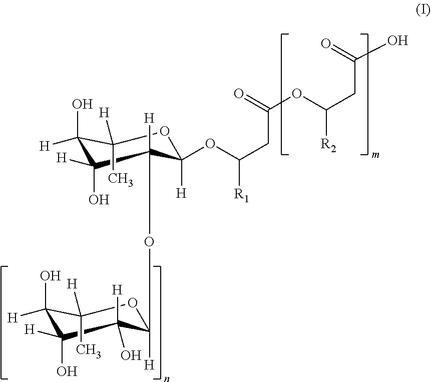
in which:
m=2, 1 or 0,
n=1, and
R₁ and R₂, independently of one another, are identical or different hydrocarbon radicals having from 2 to 24, preferably from 5 to 13 carbon atoms, where appropriate that are branched, optionally substituted, in particular hydroxy-substituted, optionally unsaturated, in particular a mono-, di- or tri-unsaturated alkyl radical,
and also the salts thereof, solvates thereof and optical isomers thereof,
for reducing and/or inhibiting the development of the colour, for example brown or black colour, of cutaneous comedones and/or preventing the appearance of cutaneous blackheads.

2. Use according to claim 1, of at least one dirhamnolipid of general formula (I), in which R₁ and R₂, which may be identical or different, are chosen from pentenyl, hexenyl, heptenyl, octenyl, nonenyl, decenyl, undecenyl, dodecenyl and tridecenyl radicals and radicals of formula —(CH₂)ₒCH₃ with o being an integer varying from 1 to 23, in particular from 3 to 15 and more particularly from 4 to 12.

3. Use according to claim 1 or 2, of at least one dirhamnolipid of general formula (I) in which m is equal to 1.

4. Use according to any one of the preceding claims, in which said dirhamnolipid of general formula (I) is used in the form of a mixture of at least two and preferably of at least three dirhamnolipids of general formula (I) in which m is preferably equal to 1.

5. Use according to any one of the preceding claims, of at least one dirhamnolipid compound, of formula (II): 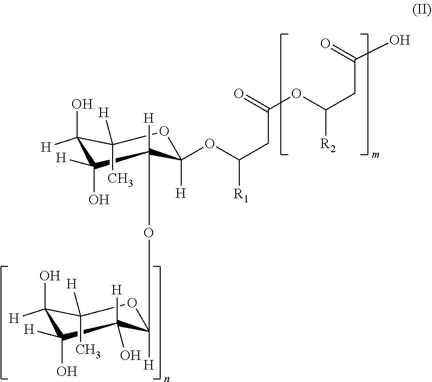
in which:
m=2, 1 or 0, and preferably m=1,
n=1,
R₁ is a —(CH₂)ₚ—CH₃ radical, with p being an integer varying from 1 to 23, preferably from 4 to 12,
R₂ is a —(CH₂)_{q}—CH₃ radical, with q being an integer varying from 1 to 23, preferably from 4 to 12,
and also the salts thereof, solvates thereof and optical isomers thereof.

6. Use according to the preceding claim, of at least one dirhamnolipid compound, of formula (II), in which p and q are identical and equal to 6 and m is equal to 1 or one of the salts, solvates and optical isomers thereof, preferably the dirhamnolipid of formula (II) in which p and q are identical and equal to 6 and m is equal to 1 is used in the form of a mixture of rhamnolipids, comprising it in a proportion of at least 50% by weight and preferably from 51% to 85% by weight relative to the total weight of rhamnolipids.

7. Use according to claim 5, of at least one dirhamnolipid compound of formula (II) in which m is equal to 1, p is equal to 6, q is equal to 8, or one of the salts, solvates and optical isomers thereof.

8. Use according to any one of claims 5 to 7, in which said dirhamnolipid of formula (I) is used in the form of a mixture of rhamnolipids comprising at least two, in particular at least three dirhamnolipids of formula (II) or of formula (I) chosen from:
a dirhamnolipid of formula (II), in which p and q are identical and equal to 6 and m is equal to 1,
a dirhamnolipid of formula (II) in which m is equal to 1, p is equal to 6, q is equal to 8, and
a dirhamnolipid of general formula (I) in which m and n are equal to 1, R₁ is a —(CH₂)ₒCH₃ radical, with o being an integer varying from 4 to 12, and R₂ is chosen from pentenyl, hexenyl, heptenyl, octenyl, nonenyl, decenyl, undecenyl, dodecenyl and tridecenyl radicals, and preferably R₁ is a —(CH₂)₆CH₃ radical and R₂ is a nonenyl radical.

9. Use according to any one of claims 5 to 8, in which said dirhamnolipid of formula (I) is used in the form of a mixture of rhamnolipids comprising at least two, in particular at least three dirhamnolipids of formula (II) or of formula (I) chosen from:
at least 50% by weight, preferably from 51% to 85% by weight of a dirhamnolipid of formula (II) in which p and q are identical and equal to 6 and m is equal to 1, relative to the total weight of rhamnolipids,
from 0.5% to 25% by weight, preferably from 5% to 15% by weight, of a dirhamnolipid of formula (II) in which p is equal to 6, q is equal to 8 and m is equal to 1, relative to the total weight of rhamnolipids, and
from 0.5% to 15% by weight, preferably from 3% to 12% by weight, preferably from 5% to 10% by weight, of a dirhamnolipid of general formula (I) in which n and m are equal to 1, R₁ is a —(CH₂)₆CH₃ radical and R₂ is a nonenyl radical, relative to the total weight of rhamnolipids.

10. Use according to any one of the preceding claims, in which said dirhamnolipid of formula (I) is used in a topical cosmetic composition in a proportion of from 0.01% to 10% by weight, preferably from 0.1% to 5% by weight, even better from 0.3% to 3% by weight of dirhamnolipid(s) of general formula (I) relative to the total weight of the cosmetic composition.

11. Use according to any one of the preceding claims, in which said cosmetic composition is intended for topical application and preferably is in the form of an oil-in-water emulsion.

12. Cosmetic method for reducing and/or inhibiting the development of the black or brown colour of cutaneous comedones and/or for preventing the appearance of blackheads, comprising:
a) a step of topical application to the skin of a washing solution;
b) a step of topical application to the skin of a cosmetic composition comprising at least one dirhamnolipid of general formula (I) or (II) according to any one of claims 1 to 11, notably in the form of an oil-in-water emulsion; and
c) preferably, a step of topical application of a composition different from the composition applied in step b) and containing at least one active agent distinct from said dirhamnolipid of general formula (I) or (II);
it being possible for steps b) and c) to be carried out simultaneously or successively.

13. Method according to the preceding claim, in which the skin is the skin of the face and/or of the body, in particular of the face and/or of the hands, preferably of the face, and more particularly of the forehead and/or of the alae of the nose and/or of the chin.
